(19) United States
(12) Patent Application Publication
(10) Pub. No.: US 2011/0256527 A1
(43) Pub. Date: Oct. 20, 2011

(54) RAPID AND SAFE TECHNIQUE FOR PERFORMING PCR AMPLIFICATIONS

(75) Inventors: Pierre J. Messier, Quebec (CA); Marc Tordjeman, Bandol (FR)

(73) Assignee: SafeLife/Triosyn Corp., Williston, VT (US)

(21) Appl. No.: 13/124,736

(22) PCT Filed: Oct. 23, 2009
(86) PCT No.: PCT/US09/05782

§ 371 Date: Jun. 21, 2011

Related U.S. Application Data
(60) Provisional application No. 61/197,260, filed on Oct. 23, 2008.

(51) Int. Cl. C12Q 1/70; C12Q 1/68

(52) U.S. Cl. 435/5; 435/6.12; 525/540

(57) ABSTRACT

This invention relates to methods for quick and safe identification of pathogens from biological samples. Iodinated resins may be employed to destroy a pathogen while leaving the pathogen's DNA in a state that can be analyzed. The DNA can then serve as a substrate for PCR analysis. The use of these iodinated resins work in a significantly quicker manner than prior art methods and allows scientists to spend a minimal time under Biosafety Level Three (BSL-3) conditions.

### FIELD OF THE INVENTION

This invention relates to a novel broad spectrum rapid preparation method for extracting DNA to be used for PCR amplification and other molecular biological processes.

### BACKGROUND OF THE INVENTION

The polymerase chain reaction (PCR) provides a method for increasing the number of copies of a target sequence (amplifying the signal) with or without having to culture the organism prior, thereby allowing increased sensitivity in detecting DNA sequences present in small amounts in samples with DNA from mixed populations (Ou, C. Y.; et. al., Science 239:292-295; Saiki, R. K.; et. al. Science 239:487-494; Saiki, R. K.; et. al. Science 230:1350-1354; Scharf, S. J.; et. al. Science 233:1076-1078). The method involves melting the DNA and annealing short oligomer primers to regions flanking a target sequence. DNA polymerase is added to the mixture in the presence of free deoxynucleotides, and DNA is extended from the primers across the target region. The new duplexes are again melted and the process is repeated. This results in the exponential accumulation of the specific target, approximately 2ⁿ, where n is the number of cycles of melting and primer extension. Single-copy genomic sequences can be amplified by a factor of more than 10 million with high specificity. The method has been refined by the use of a thermally stable polymerase isolated from Thermus aquaticus (Taq), which obviates the need to add new polymerase after each melting cycle, and has been shown to have great specificity.

An integral aspect of the fields of microbiology and the practice of medicine is the ability to positively identify microorganisms at the level of genus, species or serotype. Correct identification is not only an essential tool in the laboratory but plays a significant role in the control of microbial contamination in the processing of food stuffs, production of agricultural products and monitoring of environmental media such as ground water. Increasing stringency in regulations which apply to microbial contamination have resulted in a corresponding increase in industry resources which must be dedicated to contamination monitoring.

Owing to its ability to identify microorganisms and detect viral DNA, PCR has become increasingly important in the detection and diagnosis of diseases. Weisburg et al., (EP 517361), for example, discloses a method for the detection and identification of pathogenic microorganisms involving the PCR amplification of DNA of E. coli. Detection of bacterial and viral DNA can significantly aid in the treatment of many diseases. Accordingly, in today's medical field, it important for doctors and other staff to quickly and accurately detect the presence of pathogens in the biological or other fluids related to their patients.

Prior to performing a PCR, it is first necessary to purify the DNA (or RNA) of a sample. Purification of DNA requires an extraction procedure which generally involves disrupting the cellular membranes of samples to be analyzed, denaturing proteins in the cells and separating the DNA from the denatured protein and other cellular components. The DNA extraction techniques available to clinicians and forensic scientists have been time-consuming and laborious, often requiring multiple steps and the use of hazardous reagents. Traditional DNA extraction techniques include density gradient centrifugation, organic solvent precipitation and/or salt precipitation. Besides the aforementioned problems, these techniques increase the risk of cross-contamination from sample to sample.

The problems associated with DNA extraction and purification have resulted in newer methods designed to increase efficiency and safety. Solid phase extraction methods such as those described in U.S. Pat. No. 5,234,809 and U.S. Pat. No. 7,238,530 have been applied to the purification of nucleic acids including DNA and RNA. The method involves lysing cells, binding the released DNA to a solid support, washing away impurities and eluting the purified DNA. The preferred lysing agents are the chaotropes guanidinium thiocyanate and guanidinium hydrochloride. The solid support is preferentially a silica particle. While the method aims at reducing the manipulation of the sample, it is still microorganism specific and still requires multiple laboratory steps. Moreover, the DNA extraction process requires the use of toxic reagents at high concentrations.

Liquid phase methods have also been designed to simplify the DNA extraction process. One such method relies on using the chelating resin Chelex® 100, a styrene-divinylbenzene copolymer containing paired iminodiacetate. This resin is capable of scavenging metal contaminants (e.g. magnesium) that catalyze the degradation of nucleic acids. Additionally, Chelex® 100 is able to disrupt cell membranes and denature DNA. In practice, the sample is diluted in water in the presence of proteinase K and the solution is incubated at 55° C. for approximately one hour. The mixture is then heated to a temperature of 100° C. for another 15 minutes. The mixture is then shaken in a vortex and centrifuged. Denatured protein and metal ions settle at the bottom of the tube. An aliquot from the supernatant can be used to perform the PCR. It is noted that the process requires multiple manipulation, complex human interaction and heating to 100° C., which adds to processing time and increases processing hazards, particularly when working with dangerous pathogenic material. Furthermore, the process is not amenable to working with all types of samples.

It is further noted that the majority of kits on the market provide limited means of producing a sample that is non-pathogenic, forcing the analyst to perform the PCR experiment under biohazard conditions. This presents significant safety issues that have not been adequately addressed in the art. As such, researches may need to perform experiments in a Biosafety Level 3 or 4 (BSL-3 or BSL-4) laboratory, which further adds to processing times.

Hence, there is exists a need to develop simplified, more rapid and less hazardous procedures for analyzing biological samples. In particular, there exists a need to develop a faster, safer and more economical way to analyze pathogenic samples. Methods must be able to deactivate the pathogen rapidly and efficaciously while retain the integrity of the nucleic acid (e.g. DNA) to be amplified and analyzed. Furthermore, a simplified procedure would allow non-expensive PCR equipment to be used in small remote clinics and hospital centers to help medical personnel to obtain a fast confirmed diagnostic in order to treat their patients.

### SUMMARY OF THE INVENTION

The present invention allows for a very rapid and simple (one-step) method to lyse cells of a liquid sample (i.e. blood, saliva, urine and others) in order to extract nucleic acids (e.g. DNA or RNA) from pathogens found in the sample and simultaneously provides a biohazard free sample for the laboratory personnel to handle without complex equipment such as thermal or ultrasonic equipment. The DNA can serve as a substrate for PCR or other microbiological procedures, which results in the rapid amplification and identification of the pathogen, while at the same time destroying the viability of the pathogen in question.

In one aspect of the present invention, an iodinated resin is used as an active agent to lyse the cells of a liquid sample containing a pathogen (e.g. virus or bacteria), and destroy the pathogenic capability of the pathogen Nucleic acids from the lysed cells are then amplified and identified using a PCR procedure. The iodinated resin does not negatively impact the integrity of the DNA and hence, the performance and sensitivity of the PCR are optimal.

One aspect of the current invention is a process for analyzing a biological sample comprising the steps of: providing a liquid sample containing a pathogen, contacting the liqiud sample with an iodinated resin, separating the iodinated resin from the liquid and determining the identity of the pathogen through analyzation techniques.

Yet another aspect of the present invention is a diagnostic kit for analyzing a biological sample comprising the steps of: providing a liquid sample containing a pathogen, contacting the liqiud sample with an iodinated resin, separating the iodinated resin from the liquid and determining the identity of the pathogen through analyzation techniques.

Yet another aspect of the present invention is a Pre-PCR diagnostic kit comprising an iodinated resin, said kit allowing the elimination of thermal and/or cooling and/or sonification steps and equipment.

### DETAILED DESCRIPTION OF THE INVENTION

The present invention provides a rapid and safe method for isolating the DNA of a pathogenic sample for use in PCR or other microbiological procedures. The method involves using a demand disinfectant iodinated resin as an active agent to lyse the cell membrane of a bacterial or protective coat of a virus, thereby removing the nucleic acid material from the pathogen. The iodinated resin destroys the deleterious effects of a pathogen while preserving the DNA of said pathogen. Surprisingly, it has been found that the new protocol significantly cuts down, the number of laboratory steps and the time and labor needed to analyze biological samples with PCR, while at the same time destroying the pathogenic capabilities of the microbes in question.

An iodinated resin product has been proposed for use as a demand disinfectant, namely a disinfectant wherein iodine is released almost entirely on a demand-action basis. An iodinated resin (the “Triosyn” resin), such as the one disclosed in U.S. Pat. No. 5,639,452 (the '452 patent) can be used to enhance the lysis and destroy the pathogenicity of a microbe with no negative impact on the PCR process. The contents of the '452 patent is incorporated by reference in its entirety herein. Triosyn samples (beads, fragments, powders) all contain triiodide molecules, which are the antimicrobial components being used to disrupt the membranes of microorganisms such as viruses, bacteria and fungi. In addition to the triiodide present, fragments and powders possess another antimicrobial property which comes from their irregular shapes and their sharp edges, thus allowing the Triosyn fragments or particles to mechanically insert into the cell membrane of a bacteria or protective coat of a virus.

Three such Triosyn resin powders used in accordance with the present invention are referred to as Triosyn T-50 powder, Triosyn T-45 powder and Triosyn T-40 powder. The numbers refer to the approximate weight percentage of iodine relative to the resin. Powders with other weight percentages of iodine may also be used in accordance with the present invention.

Different percentages of iodine in the iodinated resin powders will confer different properties to the powder, in particular different levels of lysis and biocidal activity. The particular resin used in the process is based on the desired application. The amount of Triosyn resin beads in a sample being processed for PCR will be in the range of about 0.0025 grams to about 0.5 grams per 500 μL of a bacterial suspension.

In one aspect of the present invention, a rapid and safe method for purifying DNA or RNA from a liquid sample containing a pathogen is provided. The sample can be, for example, blood, saliva, or urine. The sample containing the pathogen is added to a sterile microtube. The Triosyn beads or fragments are then added and the sample is vortexed. Vortexing is done for a time between 1 minute and 5 minutes, depending on the nature of the sample and iodinated resin active agent. The sample is then centrifuged to allow the iodinated resin to migrate to the bottom of the tube. Centrifuging can be done for approximately 1 to 10 minutes, preferably about 5 minutes. Following centrifugation, the majority of the DNA and the RNA is found in the supernatant rather than at the bottom of the vessel. Likewise, impurities that would normally cause degradation of the DNA migrate to the bottom of the tube. These impurities likely include proteins which are denatured by the iodinated resin as well as metals that are exchanged with the iodinated resin. Accordingly, the DNA or RNA in the supernatant contains DNA or RNA that is viable and can be used as a substrate for further applications including PCR. The iodinated resin does not damage the DNA or RNA of the sample.

In an alternative embodiment, proteinase K may be added to the sample containing the iodinated resin prior to vortexing. The proteinase K will assist the iodinated resin in denaturing proteins that are in the sample.

Once the sample is rendered harmless by the iodinated resin, the biological sample may be safely examined by PCR analysis without further preparation or process to determine which virus or bacteria is contained in the sample. PCR amplifications are standard in the art. For example, PCR amplification procedures have been described in U.S. Pat. Nos. 5,234,809, 5,928,906, and 7,238,530, all of which are hereby incorporated by reference. Importantly, the iodinated resin does not have a negative impact on the PCR process and enzymes. Thus, using the inventive protocol, medical staff has the ability to quickly and safely determine the nature of the virus or bacteria are present in the particular biological sample. Moreover, owing to its simplicity and low cost, the process enables PCR equipment to be located in small medical centers and remote areas and allows the medical personnel to obtain rapid diagnostic information. This is not available today since current protocols require specific and complex diagnostic kits that necessitate the use of expensive thermal and or sonification equipment to be able to execute the test.

### Exemplary Microbes

Below is a nonlimiting list of microbes that can be detected by method used in accordance with the present invention.
Order Caudovirales
   Family Myoviridae—includes Enterobacteria phage T4
   Family Podoviridae
   Family Siphoviridae—includes Enterobacteria phage λ
Order Herpesviridae
   Family Alloherpesviridae
   Family Herpesviridae—includes human herpesviruses, Varicella Zoster virus
   Family Malacoherpesviridae
Unassigned families
   Family Ascoviridae
   Family Adenoviridae
   Family Asfarviridae—includes African swine fever virus
   Family Baculoviridae
   Family Coccolithoviridae
   Family Corticoviridae
   Family Fuselloviridae
   Family Guttaviridae
   Family Iridoviridae
   Family Lipothrixviridae
   Family Nimaviridae
   Family Papillomaviridae
   Family Phycodnaviridae
   Family Plasmaviridae
   Family Polyomaviridae—includes Simian virus 40, JC virus
   Family Poxviridae—includes Cowpox virus, smallpox
   Family Rudiviridae
   Family Tectiviridae
Unassigned genera
   Mimivirus
Unassigned bacteriophage families
   Family Inoviridae
   Family Microviridae
Unassigned families
   Family Geminiviridae
   Family Circoviridae
   Family Nanoviridae
   Family Parvoviridae—includes Parvovirus B19
Unassigned genera
   Anellovirus
Turreted
   Genus Aquareovirus: type species Aquareovirus A
   Genus Cypovirus: type species Cypovirus 1 (CPV 1)
   Genus Fijivirus: type species Fiji disease virus
   Genus Idnoreovirus: type species Idnoreovirus 1
   Genus Mycoreovirus: type species Mycoreovirus 1
   Genus Orthoreovirus: type species Mammalian orthoreovirus
   Genus Oryzavirus: type species Rice ragged stunt virus
Nonturreted
   Genus Coltivirus: type species Colorado tick fever virus (CTFV)
   Genus Orbivirus: type species Bluetongue virus
   Genus Phytoreovirus: type species Rice dwarf virus
   Genus Rotavirus: type species Rotavirus A—a common cause of diarrhea
   Genus Seadornavirus
Order Nidovirales
   Family Arteriviridae
   Family Coronaviridae—includes Coronavirus, SARS
   Family Roniviridae
Unassigned
   Family Astroviridae
   Family Barnaviridae
   Family Bromoviridae
   Family Caliciviridae—includes Norwalk virus
   Family Closteroviridae
   Family Comoviridae
   Family Dicistroviridae
   Family Flaviviridae—includes Yellow fever virus, West Nile virus, Hepatitis C virus, Dengue fever virus
   Family Flexiviridae
   Family Leviviridae
   Family Luteoviridae—includes Barley yellow dwarf virus
   Family Marnaviridae
   Family Narnaviridae
   Family Nodaviridae
   Family Picornaviridae—includes Poliovirus, the common cold virus, Hepatitis A virus
   Family Potyviridae
   Family Sequiviridae
   Family Tetraviridae
   Family Togaviridae—includes Rubella virus, Ross River virus, Sindbis virus, Chikungunya virus
   Family Tombusviridae
   Family Tymoviridae
   Unassigned genera
      Genus Benyvirus
      Genus Cheravirus
      Genus Furovirus
      Genus Hepevirus—includes Hepatitis E virus
      Genus Hordeivirus
      Genus Idaeovirus
      Genus Ourmiavirus
      Genus Pecluvirus
      Genus Pomovirus
      Genus Sadwavirus
      Genus Sobemovirus
      Genus Tobamovirus—includes tobacco mosaic virus
      Genus Tobravirus
      Genus Umbravirus
Order Mononegavirales
   Family Bornaviridae—Boma disease virus
   Family Filoviridae—includes Ebola virus, Marburg virus
   Family Paramyxoviridae—includes Measles virus, Mumps virus, Nipah virus, Hendra virus
   Family Rhabdoviridae—includes Rabies virus
Unassigned
   Family Arenaviridae—includes Lassa virus
   Family Bunyaviridae—includes Hantavirus
   Family Orthomyxoviridae—includes Influenza viruses
   Unassigned genera:
      Genus Deltavirus
      Genus Ophiovirus
      Genus Tenuivirus
      Genus Varicosavirus
Family Metaviridae
Family Pseudoviridae
Family Retroviridae—Retroviruses, e.g. HIV
Family Hepadnaviridae—e.g. Hepatitis B virus
Family Caulimoviridae—e.g. Cauliflower mosaic virus
   The bacteria of the following phyla:
Acidobacteria
Actinobacteria
Aquificae
Bacteroidetes/Chlorobi
Chlamydiae/Verrucomicrobia
Chloroflexi
Chrysiogenetes
Cyanobacteria
Deferribacteres
Deinococcus-Thermus
Dictyoglomi
Fibrobacteres
Firmicutes
Fusobacteria
Gemmatimonadetes
Nitrospirae
Planctomycetes
Proteobacteria
Spirochaetes
Synergistetes
Tenericutes
Thermodesulfobacteria
Thermotogae

### EXAMPLES

The following sections describe exemplary embodiments of the present invention. It should be apparent to those skilled in the art that the described embodiments of the present invention provided herein are illustrative only and not limiting, having been presented by way of example only. All features disclosed in this description may be replaced by alternative features serving the same or similar purpose, unless expressly stated otherwise. Therefore, numerous other embodiments of the modifications thereof are contemplated as falling within the scope of the present invention as defined herein and equivalents thereto.

### Experimental Data of Activity of Iodinated Resins On Pathogens

The following examples show the ability of demand disinfectant iodinated resins to eliminate the pathogenicity of microbes.

### General Procedure

1) Place an indicated amount of Triosyn in pre-labeled tubes.
2) Add 10 mL of Vanomycin-resistant enterococci (“VRE”) suspension (or mrsa, herpes virus, hepatitis virus, Chlamidia).
3) Vortex (setting 5) for indicated contact time.
4) Dilute in PBS and plate immediately (no neutralization).

### Example 1

Triosyn T40 beads (0.025 grams) were added to a pre-labeled tube into which 10 mL of a VRE suspension (one colony diluted with 300-500 μL) was added and the sample was then vortexed. The sample was analyzed at time points of 0, 2, 5, 10, 15 and 30 minutes for the presence of active VRE. It was found that at the 2 and 5 minute time points there was a reduction of the active VRE and by the 10 minute time point there was virtually no active VRE left in the tube. At the 15 and 30 minute time points there was no active VRE present. This experiment was conducted with a control that vortexed the VRE suspension with no Triosyn T40 beads present, resulting in no reduction in the amount of active VRE.

### Example 2

Triosyn T40 beads (0.025 grams) were added to a pre-labeled tube into which 10 mL of a VRE suspension (one colony diluted with 300-500 μL) was added and the sample was then vortexed. The sample was analyzed at time points of 0, 2, 5, 10, 15 and 30 minutes for the presence of active VRE. It was found that at the 0 minute time point there was a reduction of the active VRE and by the 2 minute time point there was virtually no active VRE left in the tube. At the 5, 10, 15 and 30 minute time points there was no active VRE present. This experiment was conducted with a control that vortexed the VRE suspension with no Triosyn T50 beads present, resulting in no reduction in the amount of active VRE.

### Example 3

Triosyn T40 beads (0.025 grams) were added to a pre-labeled tube into which 10 mL of a VRE suspension (one colony diluted with 300-500 μL) was added and the sample was then vortexed. The sample was analyzed at time points of 0, 2, 5, 10, 15 and 30 minutes for the presence of active VRE. It was found that at the 0 and 2 minute time points there was a reduction of the active VRE and by the 5 minute time point there was virtually no active VRE left in the tube. At the 10, 15 and 30 minute time points there was no active VRE present. This experiment was conducted with a control that vortexed the VRE suspension with no Triosyn T45 beads present, resulting in no reduction in the amount of active VRE.

### Example 4

Triosyn T40 beads (0.025 grams) were added to a pre-labeled tube into which 10 mL of a VRE suspension (one colony diluted with 300-500 μL) was added and the sample was then vortexed. The sample was analyzed at time points of 0, 2, 5, 10, 15 and 30 minutes for the presence of active VRE. It was found that at the 0 minute time point there was virtually no active VRE left in the tube, the fragments did not need to vortexed. At the 2, 5, 10, 15 and 30 minute time points there was no active VRE present. This experiment was conducted with a control that vortexed the VRE suspension with no Triosyn T50 fragments present, resulting in no reduction in the amount of active VRE.

### Example 5

Purolite A-605 iodinated resin beads (0.025 grams) were added to a pre-labeled tube into which 10 mL of a VRE suspension (one colony diluted with 300-500 μL) was added and the sample was then vortexed. The sample was analyzed at time points of 0, 2, 5, 10, 15 and 30 minutes for the presence of active VRE. It was found that at the 0 minute time point there was virtually no active VRE left in the tube, the fragments did not need to vortexed. At the 2, 5, 10, 15 and 30 minute time points there was no active VRE present. This experiment was conducted with a control that vortexed the VRE suspension with no Purolite A-605 beads present, resulting in no reduction in the amount of active VRE.

### Example 6 (Control Example)

Triosyn 402-C1 resin beads (0.025 grams) were added to a pre-labeled tube into which 10 mL of a VRE suspension (one colony diluted with 300-500 μL) was added and the sample was then vortexed. The sample was analyzed at time points of 0, 2, 5, 10, 15 and 30 minutes for the presence of active VRE. It was found that the Triosyn 402-C1 resin did not eliminate active VRE at any time point.

The results from Examples 1-5 show that the iodinated resin is capable of completely destroying the pathogen's capability to infect a host. Control resin beads (Example 6) without iodine were not capable of deactivating the microbe. Based on these findings, we tested whether the samples containing iodinated resin can serve as substrates in DNA amplifications.

### Experimental Procedure For Performing PCR Experiments

The results below show that the method of the present invention can be used to effectively perform PCR on the DNA of a pathogen. As discussed above, because the virility of the pathogen is effectively destroyed this method allows researchers to perform the PCR experiments without contamination or the need to work under stringent biohazard conditions.

### Example 7

### Fortuitim mac farland 3

In a sterile microtube, add 500 μl of a dilution at 1:500 of a suspension of mycobacterium Fortuitum mac farland 3.
Add 4 doses (approximately 0.025 grams) of T50 Triosyn iodinated resin beads.
Vortex for 2 minutes at maximum speed.
Centrifuge for 5 minutes and test 5 μl of the supernatant in PCR reaction mixture specific for mycobacteria.
   Quantification of the results of the PCR indicated that the DNA prepared by the procedure is of high quality which can be amplified and analyzed successfully. Accordingly, the iodinated resin did not have a detrimental impact on the DNA.

We suspected that the high quality of the PCR amplification was partially related to the absence of viable microbes in the PCR solution, which would negatively impact the PCR procedure. We tested the microbiological efficiency of the Triosyn T50 beads by inoculating Loewenstein Jensen tubes with the supernatant to verify the presence/absence of bacterial development after 72 hours. The samples prepared with iodinated resin beads showed no bacterial growth after 72 hours. Control samples without iodinated resin beads showed significant bacterial growth after 72 hours (nbre of colony>200).

### Example 8

### Vancomicyn resistant Enterococcus VRE

Same protocol used for mycobacteria: 4 doses T50 Triosyn iodinated resin beads, vortex 2 minutes, and PCR of supernatant
The results of this experiment showed excellent PCR data

### Example 9

### Chlamydia

Urine sample, initial centrifuge: concentrate added to 200 uL water+4 doses T50 Triosyn iodinated resin beads; vortex for 2 minutes and analyze supernatant using PCR
The results of this experiment showed excellent PCR data

### Example 10

### Herpes Virus (HSV)

Sample recovered from transport liquid.
Addition of 4 doses of T50 Triosyn iodinated resin beads, vortex 2 minutes, and PCR of supernatant
The results of this experiment showed excellent PCR data

The results shown in Examples 7-10 show that the iodinated resin is capable of completely destroying the pathogen's capability to infect a host. Additionally, the methodology allows the user to perform high quality PCR amplifications on the samples without possible infection from the pathogen. Control experiments with an Amberlite resin (non-iodinated resin) did not allow for any PCR amplification. Hence, the iodinated resin does not destroy the integrity of the microbe's DNA. Moreover, experiments can be performed without detergents, chemical additives, filtration cooling, sonification and heating, thus greatly expediting the process. Accordingly, the performance time of the PCR procedure is significantly reduced.

1. A method of analyzing a biological sample comprising the steps of:
a) providing a liquid sample containing a pathogen;
b) contacting the liquid sample with an iodinated resin;
c) separating the iodinated resin from the liquid; and
d) determining the identity of the pathogen through analyzation techniques.

2. The method of claim 1 in which the iodinated resin is a bead or fragment.

3. The method of claim 1 in which the contacting of the sample is enhanced with a Vortex apparatus.

4. The method of claim 3, wherein the separation of the liquid portion is performed using a centrifuge.

5. The method of claim 1, wherein the analyzation technique is PCR.

6. A method of analyzing a biological sample comprising the steps of:
a) providing a liquid sample containing a pathogen;
b) contacting the liquid sample with an iodinated resin;
c) centrifuging the liquid sample containing the iodinated resin;
d) removing a portion of the liquid sample;
e) adding the portion of the liquid sample to a PCR solution specific for the pathogen; and
f) quantifying the results of the PCR process.

7. The method of claim 6, wherein the pathogen is a bacteria or a virus.

8. The method of claim 6, wherein the the contacting of the sample is initiated with a Vortex apparatus.

9. A test kit for carrying out the process according to claim 1 comprising components to amplify nucleic acid.

10. A kit for analyzing a biological sample for a pathogen comprising a iodinated resin in a sample holder which contains at least enough space to add said biological sample.

11. The kit of claim 10, further comprising components to amplify nucleic acid.

12. A Pre-PCR diagnostic kit comprising an iodinated resin, said kit allowing the elimination of thermal and/or cooling and/or sonification steps and equipment.

13. A biomolecular DNA/RNA identification kit comprising iodinated resin allowing a PCR equipment to function without thermal and/or cooling and/or sonification equipment.
